(19) United States
(12) Patent Application Publication
(10) Pub. No.: US 2016/0362649 A1
(43) Pub. Date: Dec. 15, 2016

(54) DUAL-MODE SYSTEM AND METHOD FOR PROCESSING ORGANIC MATERIAL

(71) Applicant: SGC ADVISORS, LLC, Atlanta, GA (US)

(72) Inventors: Jay R. Troger, Ada, MI (US); James R. Miller, San Juan Capistrano, CA (US); Joseph E. Zuback, Camarillo, CA (US)

(21) Appl. No.: 15/120,976

(22) PCT Filed: Nov. 11, 2014
(86) PCT No.: PCT/US14/65014

§ 371 Date: Aug. 23, 2016

Related U.S. Application Data
(60) Provisional application No. 61/943,620, filed on Feb. 24, 2014.

(51) Int. Cl. C12M 1/107; C02F 11/18; C12M 1/33; C12P 5/02; C12M 1/00; C02F 3/28; C02F 11/04

(52) U.S. Cl. CPC C12M 21/04; C02F 3/286; C02F 11/18; C02F 11/04; C12P 5/023; C12M 45/20; C12M 45/02; C02F 2301/066; C02F 2303/06

(57) ABSTRACT

A dual-mode system and method is disclosed for processing organic material. In a first mode of operation, the system pre-treats an organic feedstock before digestion for improved methanization. In a second mode of operation, the system post-treats digested material to generate a useful product.

### CROSS REFERENCE TO RELATED APPLICATION

This application claims priority to U.S. Provisional Patent Application Ser. No. 61/943,620, filed Feb. 24, 2014, the disclosure of which is hereby expressly incorporated by reference herein in its entirety.

### FIELD OF THE DISCLOSURE

The present disclosure relates to processing of organic material. More particularly, the present disclosure relates to a dual-mode system and method for processing organic material.

### BACKGROUND OF THE DISCLOSURE

Organic material, such as sludge from sewage and wastewater treatment plants (WWTP), represents a serious disposal problem. This sludge generally contains a mixture of solids, commonly referred to as biosolids, and varying amounts of free water.

The large volume of cell-bound water in biosolids makes the disposal of sewage sludge containing biosolids challenging. In particular, the cost of incinerating sewage sludge is prohibitive because the cell-bound water gives biosolids a net negative lower heating value. Similarly, if sewage sludge is thermally dewatered, the process may have a net negative energy balance due to the energy required to evaporate water from the sewage sludge. Also, the cost of transporting sewage sludge is significant because the cell-bound water impacts the weight of the sludge. Usually the WWTP must pay a “tipping fee” to have another party dispose of its biosolids. Sludge containing biosolids is presently landfilled, land-applied, or dried and used as a fertilizer. However, these disposal methods may have negative environmental effects, such as the generation of undesirable odors and the contamination of soil or groundwater by living disease-causing organisms, toxic heavy metals, and/or other chemical or pharmaceutical compounds contained in the biosolids. Between approximately 7.1 and 7.6 million dry (short) tons of biosolids are produced each year in the U.S. alone. Thus, an adequate disposal method is important.

In addition to the current need for an adequate method of disposing of biosolids, there is growing public support for increased utilization of renewable, or “green”, energy sources. Well-known forms of renewable energy include solar energy, wind energy, and geothermal energy, but these sources lack an adequate supply. Biomass materials, such as mill residues, agricultural crops and wastes, and industrial wastes, have long been used as renewable fuels. Biosolids, on the other hand, have not previously been considered as a renewable energy source due to the large volume of cell-bound water contained therein. As discussed above, the large volume of cell-bound water in biosolids significantly impacts both the cost of incinerating biosolids and the cost of transporting biosolids.

Accordingly, new systems and methods for processing and disposing of organic material are needed.

### SUMMARY

The present disclosure provides a dual-mode system and method for processing organic material. In a first mode of operation, the system pre-treats an organic feedstock before digestion for improved methanization. In a second mode of operation, the system post-treats digested material to generate a useful product.

According to an embodiment of the present disclosure, a dual-mode system is provided for processing an organic material. The system includes a digester that digests the organic material, the digester having an input and an output, and a thermal input device that heats the organic material to produce a thermally treated material, the thermal input device selectively communicating with the input of the digester in a first mode to direct the thermally treated material to the digester, the thermal input device selectively communicating with the output of the digester in a second mode to heat the digested material from the digester.

According to another embodiment of the present disclosure, a dual-mode system is provided for processing an organic material. The system includes a digester that produces a digested material, a first thermal input device operable in a first mode to heat the organic material, produce a thermally treated material, and supply the thermally treated material to the digester, and a second thermal input device operable in a second mode to heat the digested material from the digester and produce a useful product.

According to yet another embodiment of the present disclosure, a method is provided for processing an organic material. The method includes the steps of: pre-treating the organic material by heating and pressurizing the organic material in a system, the pre-treating step generating a pre-treated material; digesting the pre-treated material to produce methane, the digesting step generating a digested material; and post-treating the digested material by heating and pressurizing the digested material in the system to produce a useful product.

### BRIEF DESCRIPTION OF THE DRAWINGS

The above-mentioned and other features and advantages of this disclosure, and the manner of attaining them, will become more apparent and the invention itself will be better understood by reference to the following description of embodiments of the invention taken in conjunction with the accompanying drawings, wherein:

FIG. 1 is a schematic diagram of a system operating in a first mode to pre-treat an organic feedstock before digestion for improved methanization;

FIGS. 2A-2C are schematic diagrams of a system operating in a second mode to post-treat digested material to generate useful products; and

FIG. 3 is a schematic diagram of a system operating in both the first mode and the second mode.

Corresponding reference characters indicate corresponding parts throughout the several views. The exemplifications set out herein illustrate exemplary embodiments of the invention and such exemplifications are not to be construed as limiting the scope of the invention in any manner.

### DETAILED DESCRIPTION

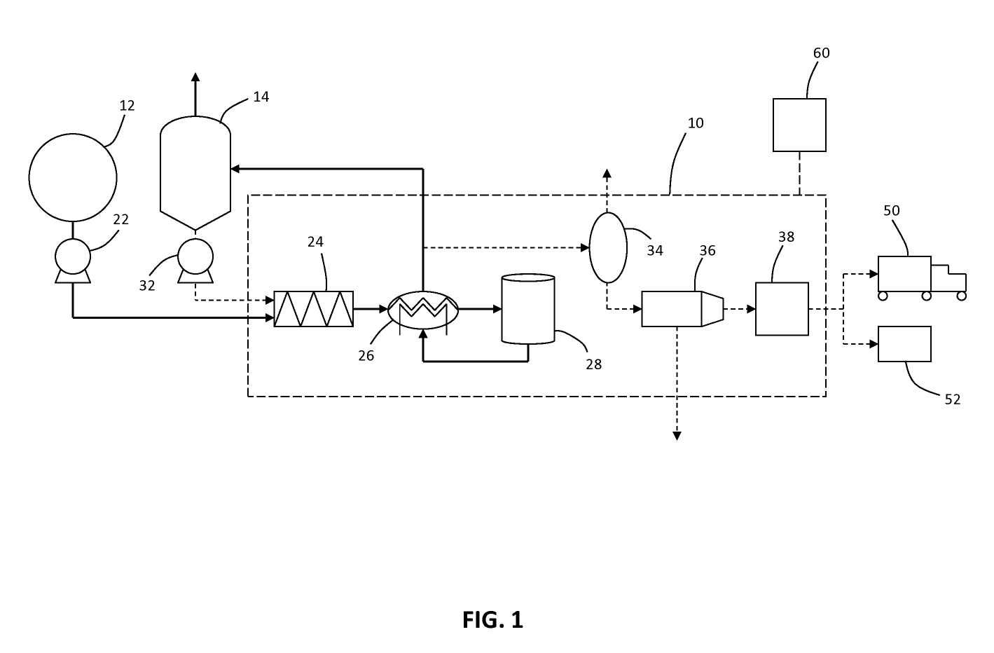

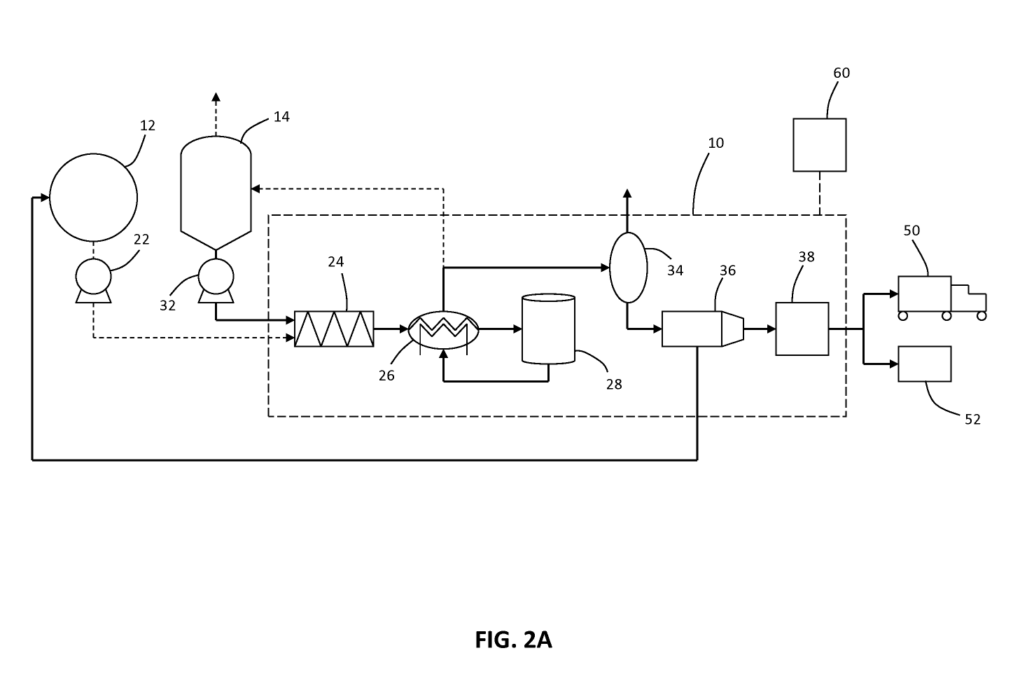

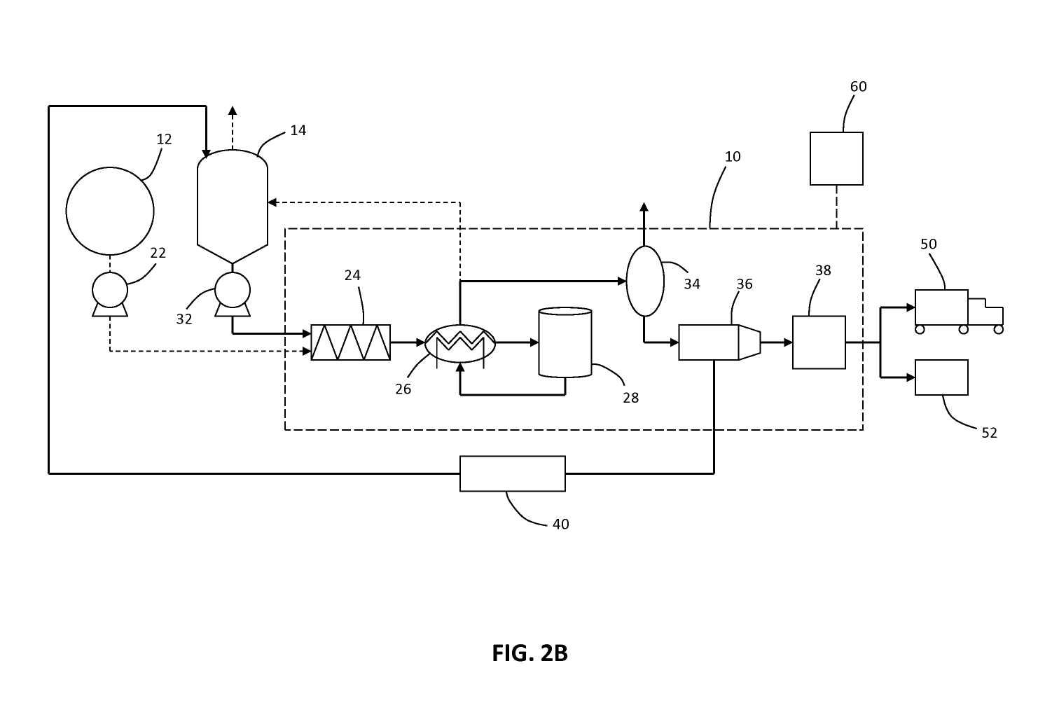

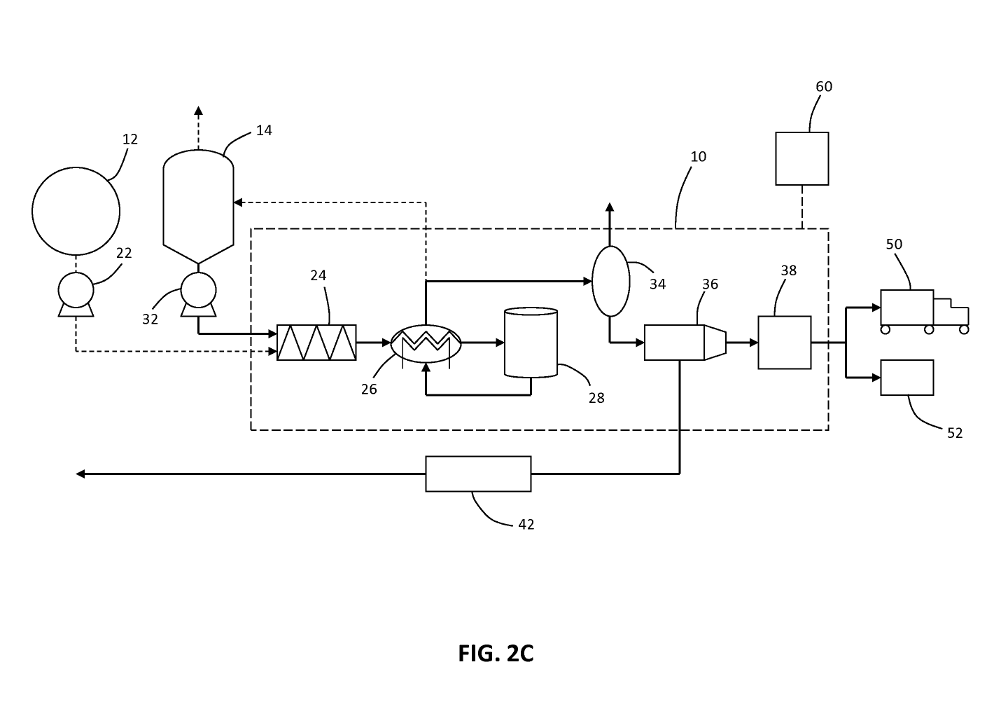

A dual-mode system 10 is disclosed for processing an organic feedstock received from a client, such as wastewater treatment plant (WWTP) 12. In a first mode of operation, which is shown in FIG. 1, system 10 pre-treats the organic feedstock from WWTP 12 and then directs the pre-treated feedstock to digester 14 for improved methanization. In a second mode of operation, which is shown in FIGS. 2A-2C, the same system 10 further treats the digested material from digester 14 to generate a useful product. The first and second modes of operation are described further below.

The first mode of operation will be described with reference to FIG. 1. Conduits that are in use during the first mode of operation are shown with solid lines in FIG. 1, and conduits that are not in use during the first mode of operation are shown with dashed lines in FIG. 1.

System 10 receives the organic feedstock from WWTP 12, which may include sewage in the form of a sludge. More specifically, the organic feedstock from WWTP 12 may include untreated sewage sludge or processed sewage sludge, such as sludge containing Class A or Class B biosolids. The term “biosolids” as used throughout this disclosure has its ordinary meaning in the art. For example, biosolids include dead organic cells, bacterial cell masses, inorganic compounds (e.g., grits, sand), cell-bound water, soil-like residue of materials removed from sewage during the wastewater treatment process, and other solids.

Prior to leaving WWTP 12, the organic feedstock may be macerated to reduce the size of solid particles contained therein. Also, the organic feedstock may be subjected to a mechanical dewatering process, such as centrifuging, belt pressing, or rotary pressing. Additionally, the organic feedstock may be subjected to a polymer treatment process, a chemical treatment process, such as being mixed with a chelating agent, or a biological treatment process, such as being mixed with bacteria and protozoans to produce waste activated sludge (WAS). Even after undergoing maceration, dewatering, and/or another pretreatment, the organic feedstock received from WWTP 12 may still include a significant amount of cell-bound water. The moisture content of the incoming organic feedstock from WWTP 12 may be as low as approximately 65 vol. %, 70 vol. %, 75 vol. %, or 80 vol. % and as high as approximately 85 vol. %, 90 vol. %, 95 vol. %, or 97 vol. %, or within any range defined between any pair of the foregoing values, for example. The remaining volume of the organic feedstock may comprise biosolids, such as dead organic cells, bacterial cell masses, inorganic compounds (e.g., grits, sand), and other solids, as well as dissolved substances, such as ammonia (NH₃).

In addition to sewage sludge, the organic feedstock from WWTP 12 may include other organic materials, especially those containing cell-bound water. For example, the organic feedstock may include paper mill sludge, food waste, plant matter (e.g., rice hulls, hay straw), discarded cellulosic packaging material, bagasse, green waste (e.g., leaves, clippings, grass), algae, wood and wood waste, clinker or other residue from combustion of wood, palm oil residue, and short rotation crops. The organic feedstock may also include animal carcasses. The organic feedstock may also include agricultural waste such as sewage material obtained from the live stock industry (e.g., hog manure, chicken litter, cow manure). The organic feedstock may also include crops grown specifically for use in the process, such as switch grass or other plants. The organic feedstock may also include municipal solid waste, fats, oils, and greases (FOG), medical waste, paper waste, refuse derived fuels, Kraft Mill black liquor, or hydrophilic non-renewable fuels (e.g., low-rank coals). In an exemplary embodiment, the organic feedstock may include a blend of biosolids and other organic materials, including biomass, to enhance the heating value of the final product and/or increase the scale of production.

In the illustrated embodiment of FIG. 1, the organic feedstock is in the form of a slurry that is pumped from WWTP 12 to system 10 using pump 22. It is also within the scope of the present disclosure that the organic feedstock may be transported from WWTP 12 to system 10 via a truck, train, barge, or another suitable mode of transport.

To prepare the organic feedstock for subsequent heating, pump 22 pressurizes the organic feedstock to a pressure above the saturation pressure of water at the subsequent elevated temperature. Pressurizing the organic feedstock maintains a liquid phase in the slurry during subsequent heating by maintaining water in the slurry below the saturated steam curve during subsequent heating and substantially inhibiting water in the slurry from vaporizing. Depending on the subsequent elevated temperature, pump 22 may pressurize the organic feedstock to a pressure as low as approximately 10 psig, 30 psig, or 50 psig and as high as approximately 1000 psig, 1300 psig, 1500 psig, or more, or within any range defined between any pair of the foregoing values, for example.

The pressure supplied by pump 22 may vary depending on the viscosity of the organic feedstock. As the viscosity of the organic feedstock increases, the pressure supplied by pump 22 may be increased to account for downstream pressure loss. Care must be exercised to provide pump 22 with an adequate net pump suction head (NPSH), either hydraulically or by mechanical assistance, considering that the organic feedstock may be very viscous and may carry dissolved gases. In one embodiment, the pressurized organic feedstock may travel from pump 22 along a vertical or downward-sloping plane to, with assistance from the Earth's gravitational force, reduce the demand on pump 22 and/or reduce the likelihood of gritty or sticky solid portions of the organic feedstock collecting downstream.

The pressurized slurry from pump 22 continues to agitator 24, as shown in FIG. 1. Agitator 24 may blend, macerate, and/or slurry the pressurized slurry to promote even heating downstream. In one embodiment, agitator 24 includes static obstacles (e.g., a static coil, static inward protrusions) to create turbulence in the slurry flowing therethrough. In another embodiment, agitator 24 includes mechanically-driven obstacles (e.g., a rotating screw, a wiping blade) to create turbulence in the slurry flowing therethrough.

Next, the pressurized slurry from agitator 24 continues to heat exchanger 26, as shown in FIG. 1, or to another suitable heater or thermal input device. In one embodiment, the pressurized slurry is heated by exchange with hot heat transfer fluid. In another embodiment, and as shown in FIG. 1, the pressurized slurry is heated by exchange with the hot slurry exiting reactor 28, which is discussed further below. Although a single heat exchanger 26 is illustrated in FIG. 1, it is within the scope of the present disclosure to heat the pressurized slurry in stages using more than one heat exchanger.

According to an exemplary embodiment of the present disclosure, heat exchanger 26 heats the pressurized slurry to a temperature sufficient to cause cellular lysing. In certain embodiments, cellular lysing begins at a temperature of about 230° F. (110° C.). At this lysing temperature, cellular structures (e.g., cellular walls, cellular lipid-bilayer membranes, internal cellular membranes) in the slurry begin to rupture. As a result, the cells begin to break down into particles of smaller size and release their cell-bound water. Also, the viscosity of the heated slurry may decrease substantially. Additionally, impurities (e.g., sodium, potassium, chlorine, sulfur, nitrogen, toxic metals) may separate from the ruptured cells as ions and dissolve into the liquid phase, making the impurities accessible for subsequent removal and disposal. To achieve such results, heat exchanger 26 may heat the pressurized slurry to a temperature as low as 230° F. (110° C.), 240° F. (116° C.), or 250° F. (121° C.) and as high as 260° F. (127° C.), 270° F. (132° C.), 280° F. (138° C.), or more, or within any range defined between any pair of the foregoing values, for example.

The pressurized and heated slurry from heat exchanger 26 is then directed to reactor 28, as shown in FIG. 1. Inside reactor 28, the heated slurry is allowed to dwell at the lysing temperature to encourage more cells to rupture and release more cell-bound water. Depending on the desired degree of cellular lysing, the residence time in reactor 28 may be as low as 1 minute, 3 minutes, or 5 minutes and as high as 7 minutes, 9 minutes, 11 minutes, or more, or within any range defined between any pair of the foregoing values, for example.

Reactor 28 may receive the heated slurry continuously (e.g., a continuous stirred-tank reactor (CSTR)) or in separate batches. Also, the heated slurry may flow downward through reactor 28 to enhance the removal of sand, grit, and other materials from the slurry, which will collect in the bottom of reactor 28. Reactor 28 may accommodate addition of an alkali, a reducing gas, or another compound to facilitate downstream removal of undesirable constituents. For example, reactor 28 may accommodate the addition of carbon monoxide to facilitate downstream removal of precipitated NH₃.

If necessary to reach or maintain the lysing temperature, additional heat may be supplied to the contents of reactor 28. For example, reactor 28 may be insulated with a jacket that receives hot heat transfer fluid to heat the contents of reactor 28. The contents of reactor 28 could also be heated by direct steam injection, heating coils, or a combination thereof, for example. It is within the scope of the present disclosure that the slurry will generate heat in reactor 28, thereby reducing or eliminating the need for additional heating of reactor 28.

The slurry leaving reactor 28, referred to herein as pre-treated slurry, contains a mixture of liquid and solid materials. The liquid phase of the pre-treated slurry includes the once-cell-bound water that was released during lysing and dissolved compounds, including dissolved carbon dioxide, dissolved NH₃, dissolved mercury, and dissolved sulfur compounds. Volatile materials, such as carbon dioxide, may be forced to remain in the liquid phase under the high pressure supplied by pump 22. However, some gases may form in the process. To prevent the evolved gases from accumulating in the piping and equipment, the evolved gases may be continuously removed from vents located throughout system 10. For example, vents may be located in reactor 28, at high points in system 10, and in confined areas, such as centrifugal pump casings, having localized pressure drops that allow dissolved gases to evolve from the liquid slurry. The solid phase of the pre-treated slurry includes primarily ruptured cellular structures and inorganic compounds (e.g., grit, sand). The solid content of the pre-treated slurry may be as low as approximately 10 wt. %, 20 wt. %, or 30 wt. %, and as high as approximately 40 wt. % or 50 wt. %, or within any range defined between any pair of the foregoing values, for example. The solid content of the pre-treated slurry may decrease in reactor 28 due to the release of bound organics into the liquid and gaseous phases, as well as chemical reactions among the constituents.

The pre-treated slurry from reactor 28 continues to heat exchanger 26, as shown in FIG. 1, or to another suitable cooler. In one embodiment, the pre-treated slurry is cooled by exchange with plant cooling water. In another embodiment, and as shown in FIG. 1, the pre-treated slurry is cooled by exchange with the cool, incoming organic feedstock. Although a single heat exchanger 26 is illustrated in FIG. 1, it is within the scope of the present disclosure to cool the pre-treated slurry in stages using more than one heat exchanger.

From heat exchanger 26, the cool, pre-treated slurry is directed to digester 14, as shown in FIG. 1. In digester 14, organic compounds in the pre-treated slurry are converted to a methane-rich gas, normally referred to as biogas. The cellular lysing that occurs in reactor 28 makes the organic compounds in the slurry more accessible to bacteria in digester 14, which increases the rate of digestion, increases the volume of biogas produced, and decreases the volume of solid sludge remaining. Increasing the rate of digestion may reduce the residence time required in digester 14, thereby freeing digester 14 to receive more material. Although one digester 14 is illustrated in FIG. 1, it is within to scope of the present disclosure to provide more than one digester.

The digestion process may occur in an anaerobic (i.e., oxygen-free) environment or in an aerobic (i.e., oxygen-rich) environment. In an exemplary embodiment, the digestion process is a two-step anaerobic process, whereby two classes of bacteria are used to convert the organic compounds in the pre-treated slurry into the biogas. First, a class of bacteria known as acidfiers or acidfying bacteria may be used to hydrolyze the complex organics into volatile fatty acids, such as acetic acid or propionic acid. Second, a class of bacteria known as methanogens may be used to convert the volatile fatty acids into the biogas.

Depending on the composition of the pre-treated slurry, the biogas generated in digester 14 may have a methane concentration between about 50 vol. % percent and 70 vol. %, with the balance comprising primarily carbon dioxide. The biogas generated in digester 14 may be used as fuel within system 10. The biogas generated in digester 14 may also be refined, such as by amine adsorption, pressure swing adsorption, or water wash, to remove carbon dioxide, water, and hydrogen sulfide, thereby producing a purified methane stream known as natural gas. The natural gas may be sold and used in industrial boilers and furnaces, for example.

The second mode of operation will now be described with reference to FIGS. 2A-2C. In this second mode of operation, the digested material from digester 14 is recirculated through system 10 to generate a useful product. Conduits that are in use during the second mode of operation are shown with solid lines in FIGS. 2A-2C, and conduits that are not in use during the second mode of operation are shown with dashed lines in FIGS. 2A-2C.

The digested material that is output from digester 14 is first re-pressurized with pump 32. Pump 32 pressurizes the digested material to a pressure above the saturation pressure of water at the subsequent elevated temperature. Pressurizing the digested material maintains a liquid phase in the slurry during subsequent heating by maintaining water in the slurry below the saturated steam curve during subsequent heating and substantially inhibiting water in the slurry from vaporizing. Depending on the subsequent elevated temperature, pump 32 may pressurize the digested material to a pressure as low as approximately 10 psig, 30 psig, or 50 psig and as high as approximately 1000 psig, 1300 psig, 1500 psig, or more, or within any range defined between any pair of the foregoing values, for example. If the subsequent elevated temperature of the second mode is higher than that of the first mode, pump 32 may pressurize the digested material to a higher pressure in the second mode than pump 22 pressurized the organic feedstock in the first mode (FIG. 1).

The pressurized slurry from pump 32 is then recirculated through agitator 24, heat exchanger 26, and reactor 28. These components of system 10 are discussed above with respect to the first mode (FIG. 1). These components may be operated in substantially the same way in the first and second modes to achieve additional or more complete cellular lysing, to the extent that complete cellular lysing was not achieved in the first mode of operation. Alternatively, these components may be operated more aggressively in the second mode to achieve more significant cellular modifications than cellular lysing. For example, these components may be operated to achieve varying degrees of decarboxylation, where oxygen splits off from the ruptured cells in the form of carbon dioxide, and/or carbonization, where the ruptured cells carbonize into char.

In one embodiment, the digested and pressurized slurry is heated to a higher temperature in the second mode than in the first mode. For example, heat exchanger 26 may heat the digested and pressurized slurry to a temperature as low as 300° F. (149° C.), 350° F. (177° C.), 400° F. (204° C.), or 450° F. (232° C.) and as high as 500° F. (260° C.), 550° F. (288° C.), or 600° F. (316° C.), or within any range defined between any pair of the foregoing values. At the higher temperature of the second mode, the digested and pressurized slurry may undergo more complete lysing than in the first mode. Additionally, the digested and pressurized slurry may undergo decarboxylation and/or carbonization at the higher temperature of the second mode.

In another embodiment, the digested and pressurized slurry is subjected to a longer residence time in reactor 28 in the second mode than in the first mode. For example, the residence time in reactor 28 during the second mode may be as low as 7 minutes, 9 minutes, or 11 minutes, and as high as 13 minutes, 15 minutes, 17 minutes, or more, or within any range defined between any pair of the foregoing values. At the longer residence time of the second mode, the digested and pressurized slurry may undergo more complete lysing than in the first mode. Additionally, the digested and pressurized slurry may undergo decarboxylation and/or carbonization at the higher residence time of the second mode.

The slurry leaving reactor 28, referred to herein as post-treated slurry, contains a mixture of liquid and solid materials. Depending on the degree of lysing, decarboxylation, and carbonization achieved in reactor 28 during the first and second modes and the degree of digestion achieved in digester 14, the solid phase may include char, ruptured cellular structures, and inorganic compounds (e.g., grit, sand). The solid particles in the post-treated slurry may have various sizes. In an exemplary embodiment, approximately 95% or more of the solid particles in the post-treated slurry are less than 150 μm or 200 μm in size. Also, approximately 50% or more of the solid particles in the post-treated slurry are less than 10 μm, 20 μm, or 50 μm in size.

The post-treated slurry may be significantly reduced in viscosity compared to the incoming organic feedstock. For example, while the incoming organic feedstock may have a viscosity of 10,000 cp or more, the post-treated slurry may have a viscosity as low as approximately 30 cp, 100 cp, or 170 cp or as high as approximately 1200 cp, 1650 cp, or 2600 cp, or within any range defined between any pair of the foregoing values.

The post-treated slurry from reactor 28 continues to heat exchanger 26, as shown in FIGS. 2A-2C, or to another suitable cooler. In one embodiment, the post-treated slurry is cooled by exchange with plant cooling water. In another embodiment, and as shown in FIGS. 2A-2C, the post-treated slurry is cooled by exchange with the cool, digested material from digester 14. Although a single heat exchanger 26 is illustrated, it is within the scope of the present disclosure to cool the post-treated slurry in stages using more than one heat exchanger.

After being cooled in heat exchanger 26, the post-treated slurry continues to pressure letdown tank 34. In pressure letdown tank 34, the pressure of the post-treated slurry may be reduced to atmospheric pressure, 5 psig, or 10 psig, for example. The pressure reduction liberates volatile materials once forced to remain in the liquid phase, such as carbon dioxide, hydrogen sulfide, and other non-condensable gases. The pressure reduction may also liberate some small amounts of water vapor. However, by cooling the post-treating slurry before depressurization, most of the water will remain in the liquid phase for removal during subsequent mechanical dewatering and thermal drying processes. Stated differently, the water may stay below the saturated steam curve during cooling and subsequent depressurization. Pressure letdown tank 34 may also be used to release vent gases that evolved elsewhere in system 10. For example, vent piping (not shown) may connect reactor 28 and/or digester 14 to pressure letdown tank 34 to release gases that evolved in reactor 28 and/or digester 14, along with the other gases that evolved in pressure letdown tank 34.

It is also within the scope of the present disclosure to depressurize the post-treated slurry in pressure letdown tank 34 without first cooling the post-treated slurry in heat exchanger 26. In this embodiment, the hot slurry from reactor 28 travels directly to pressure letdown tank 34 without traveling through heat exchanger 26. Thus, the post-treated slurry may enter pressure letdown tank 34 at the elevated temperature of reactor 28. The pressure reduction causes the slurry and water vapor to simultaneously cool to the saturation temperature of the liquid at the reduced pressure. Therefore, it may be advantageous to recover the heat lost from the slurry directly from pressure letdown tank 34.

The simultaneous reduction in pressure and temperature described in the previous paragraph, referred to as a flash separation, liberates volatile materials once forced to remain in the liquid phase, such as carbon dioxide, hydrogen sulfide, mercaptans, and other non-condensable gases, as well as water vapor. Because NH₃ exists in equilibrium with water in the slurry, NH₃ may also evaporate along with the water vapor. Evaporating NH₃ may make the final product more suitable for subsequent combustion and may allow the evaporated NH₃ to be recovered, such as with an ammonia scrubber, and sold.

The outputs from pressure letdown tank 34 include a liberated vapor stream and a solid-liquid slurry stream. The liberated vapor stream exiting pressure letdown tank 34 may be captured, purified, and sold, burned to destroy odors, burned for energy recovery, processed to destroy undesirable components, or otherwise processed. The solid-liquid slurry stream is directed to a mechanical dewatering device, illustratively centrifuge 36. Other suitable dewatering devices include filters, belt presses, rotary presses, and piston-type presses, such as Bucher presses, for example. The slurry entering centrifuge 36 includes primarily liquid materials, with solid materials making up as little as approximately 5 wt. %, 10 wt. %, 15 wt. %, or 20 wt. % of the slurry and as much as approximately 25 wt. %, 30 wt. %, 35 wt. %, or 40 wt. % of the slurry, or within any range defined between any pair of the foregoing values, for example. In centrifuge 36, the slurry is subjected to high speed rotation to separate the liquid materials from the solid materials. Most of the liquid materials will exit centrifuge 36 in the liquid centrate stream, and most of the solid materials will exit centrifuge 36 in the cake.

The liquid centrate stream exiting centrifuge 36 may undergo subsequent processing. In the illustrated embodiment of FIG. 2A, the liquid centrate is returned to WWTP 12 for further processing. In the illustrated embodiment of FIG. 2B, the liquid centrate is directed to ammonia removal system 40 and is then returned to digester 14 for further methanization. In the illustrated embodiment of FIG. 2C, the liquid centrate is directed to purification or filtration system 42 before being discharged from system 10 for a beneficial use. It is also within the scope of the present disclosure that the liquid centrate may be recycled elsewhere into system 10 or that the liquid centrate may be treated for other uses.

The cake exiting centrifuge 36 may contain essentially equal amounts of solid and liquid materials. For example, the solid content of the cake may be as low as approximately 30 wt. %, 35 wt. %, 40 wt. %, or 45 wt. % and as high as approximately 50 wt. %, 55 wt. %, 60 wt. %, 65 wt. %, or more, or within any range defined between any pair of the foregoing values. The cake may constitute a useful product, with or without requiring further processing. In some embodiments, the cake may be land-applied and used as a fertilizer without requiring further processing. In other embodiments, the cake may continue to a thermal dryer 38 to drive off more water and other volatile materials to produce a renewable fuel product, for example. Dryer 38 may be powered using biogas from digester 14 as fuel, for example.

The outputs from dryer 38 include a dried solid fuel product and vaporized products, such as water vapor, that were driven off from the solid fuel product. Dryer 38 may be controlled to optimize the dried solid fuel product for its ultimate use. When the dried solid product will be used as a combustion fuel, for example, dryer 38 may be controlled to produce particles of a desired size for combustion, wherein the particles are large enough to prevent dusting, yet small enough to provide a high surface area for easy combustion. Similarly, when the dried solid product will be used for gasification, dryer 38 may be controlled to produce particles of a desired size for gasification.

The dried solid product may include macroscopic particles formed from agglomerates of smaller, microscopic particles weakly bound together. The macroscopic particles may have a diameter as small as approximately 1 mm, 3 mm, or 5 mm and as large as approximately 1 cm, 3 cm, or more, or within any range defined between any pair of the foregoing values, for example. The density of the macroscopic particles may be as low as approximately 0.5 g/cm³, 1.0 g/cm³, or 1.5 g/cm³ and as high as approximately 2.0 g/cm³, 2.5 g/cm³, or 3.0 g/cm³, or within any range defined between any pair of the foregoing values, for example. However, the macroscopic particles may be present in various sizes and densities. The microscopic particles that bind together to form the larger, macroscopic particles may be as small as approximately 0.1 μm, 1 μm, 3 μm, 5 μm, or 10 μm in size and as large as approximately 50 μm, 75 μm, 100 μm, 125 μm, or 150 μm in size, or within any range defined between any pair of the foregoing values, for example. The microscopic particles in the dried solid fuel product may have an essentially even size distribution. If the particles are too large, they may be crushed or otherwise processed to produce particles of smaller size. If the particles are too small, they may be recycled through the process and mixed with incoming solids.

The dried solid fuel product exiting dryer 38 may contain primarily solids, with solids making up more than approximately 50 wt. % of the dried solid fuel product. For example, solids may make up as little as approximately 50 wt. %, 55 wt. %, 60 wt. %, 65 wt. %, or 70 wt. % of the dried solid fuel product and as much as approximately 75 wt. %, 80 wt. %, 85 wt. %, 90 wt. %, 95 wt. %, 97 wt. %, or 98 wt. % of the dried solid fuel product. Therefore, the dried solid fuel product exiting dryer 38 may have a moisture content as low as approximately 2 wt. %, 3 wt. %, 5 wt. %, 10 wt. %, or 15 wt. % and as high as approximately 20 wt. %, 25 wt. %, or 30 wt. %. This percentage of solid materials in the dried solid fuel product is significantly higher than the percentage of solid materials in the incoming organic feedstock from WWTP 12.

Optionally, the dried solid fuel product from dryer 38 may be blended with other fuel products, such as biomass, to increase the heating value of the solid fuel product. Then, the solid fuel product may be output from system 10 to a customer for use as a renewable fuel. In one embodiment, the dried solid fuel product is delivered to the customer via truck 50 or another suitable mode of transport for combustion. In another embodiment, the dried solid fuel product is delivered to the customer for use in a waste-to-energy process, such as gasifier 52.

Advantageously, the solid fuel product of the present disclosure has a high burn rate relative to other fuels, including coal. The high burn rate may be attributed to the oxygen content of the solid fuel product, the small particle size, and/or a catalytic contribution from one or more of the inorganic species present. When combusted as a fuel source, the solid fuel product releases energy over a short amount of time, resulting in a quick, hot flame. If the solid fuel product is gasified, the high burn rate may allow for smaller sized equipment.

Also advantageously, the solid fuel product is less sticky than other biosolid products, making the solid fuel product easier to process, transport, and use. Although the solid fuel product may include sticky, tar-like materials, these sticky materials may be covered or encapsulated by non-sticky, char carbon materials.

A controller 60 is provided to selectively operate system 10 in either the first mode or the second mode. Controller 60 may be a suitably programmed general purpose computer. In an exemplary embodiment, controller 60 operates system 10 predominantly in the first mode to supply pre-treated organic material from reactor 28 to digester 14. When digester 14 reaches its maximum or threshold capacity, for example, controller 60 may switch system 10 from the first mode of operation to the second mode of operation, where digester outputs organic material to reactor 28 for fuel production. Controller 60 may operate system 10 in the second mode as necessary to remove material from digester 14, and when enough space becomes available in digester 14, controller 60 may return system 10 to the first mode to add material to digester 14.

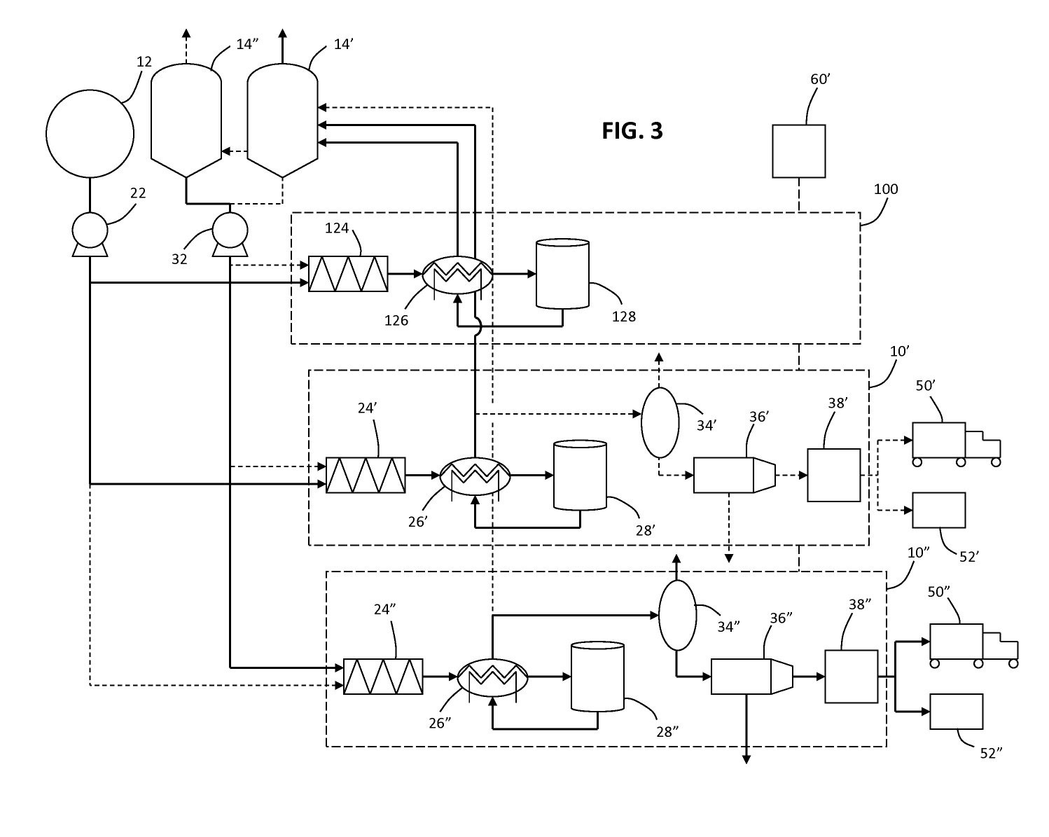

Referring next to the illustrated embodiment of FIG. 3, multiple dual-mode systems 10′, 10″ and a single-mode system 100 are included in the same process. The illustrated process of FIG. 3 also includes multiple digesters 14′, 14″, which may be in communication with each other. Thus, the material in one digester 14′ may be transferred to the other digester 14″, such as to facilitate digestion in stages.

System 10′ is shown operating in the first mode in FIG. 3, wherein system 10′ pre-treats the organic feedstock from WWTP 12 and then directs the pre-treated feedstock to digester 14′ to generate biogas. The other dual-mode system 10″ is shown operating in the second mode in FIG. 3, wherein system 10″ treats the digested material from digester 14″ to generate a renewable fuel product. By providing more than one dual-mode system 10′, 10″, and more than one digester 14′, 14″, the process is able to operate simultaneously in the first mode and the second mode to achieve higher methane and fuel production. For example, while digester 14′ is digesting the pre-treated feedstock from reactor 28′ to produce biogas, digester 14″ may simultaneously deliver digested material to reactor 28″. When digestion in digester 14′ is complete, the digested material may be transferred to digester 14″ for more digestion or to reactor 28″ for fuel production. Controller 60′ is provided to selectively operate the dual-mode systems 10′, 10″.

The process of FIG. 3 also includes a single-mode or dedicated system 100. System 100 of FIG. 3 is dedicated to the first mode of operation, wherein system 100 pre-treats the organic feedstock from WWTP 12 and then directs the pre-treated feedstock to digester 14′ to generate biogas. Because system 100 only operates in the first mode, the single-mode system 100 may require less equipment than the dual-mode systems 10′, 10″. For example, the single-mode system 100 need not include a centrifuge or a dryer. Although system 100 is dedicated to the first mode of operation, it is also within the scope of the present disclosure to include a single-mode system that is dedicated to the second mode of operation. In addition to controlling the dual-mode systems 10′, 10″, controller 60′ may also control the single-mode system 100.

Although suitable conduits are illustrated in FIGS. 1-3 for transporting organic material through dual mode systems 10, 10′, 10″, and single-mode system 100, conduits may be added or removed from the processes to affect the desired material flows. For example, in FIG. 3, the illustrated conduits direct the pre-treated slurry from reactor 28′, 28″, 128, to digester 14′. Conduits may be added to direct this pre-treated slurry to digester 14″, in addition to or instead of digester 14′.

While this invention has been described as having exemplary designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A dual-mode system for processing an organic material, the system comprising:
a digester that digests the organic material, the digester having an input and an output; and
a thermal input device that heats the organic material to produce a thermally treated material, the thermal input device selectively communicating with the input of the digester in a first mode to direct the thermally treated material to the digester, the thermal input device selectively communicating with the output of the digester in a second mode to heat the digested material from the digester.

2. The system of claim 1, further comprising a second thermal input device in communication with the digester, the thermal input devices operating in opposite modes such that:
when the thermal input device of claim 1 is operating in the first mode, the second thermal input device is operating in the second mode; and
when the thermal input device of claim 1 is operating in the second mode, the second thermal input device is operating in the first mode.

3. The system of claim 2, further comprising a third thermal input device in communication with the digester, the third thermal input device being dedicated to operate in the first mode.

4. The system of claim 1, wherein the thermal input device selectively communicates with a dewatering device in the second mode to dewater the digested material from the digester.

5. The system of claim 4, wherein the thermally treated material from the thermal input device is cooled before being directed to the digester in the first mode and the dewatering device in the second mode.

6. The system of claim 5, wherein the thermally treated material from the thermal input device is cooled by returning through the thermal input device.

7. The system of claim 4, further comprising a pressure letdown device downstream of the thermal input device and upstream of the dewatering device, wherein the thermal input device selectively communicates with the pressure letdown device in the second mode to depressurize the digested material from the digester.

8. The system of claim 1, wherein the thermal input device receives the organic material from a wastewater treatment plant in the first mode and from the digester in the second mode.

9. The system of claim 1, wherein the thermally treated material that is directed from the thermal input device to the input of the digester in the first mode is pressurized.

10. The system of claim 1, wherein the thermal input device heats the organic material to an elevated temperature, and wherein the organic material that is delivered to the thermal input device in the first and second modes is pressurized above the vapor pressure of water at the elevated temperature.

11. The system of claim 1, further comprising a controller that selectively operates the system in the first mode or the second mode.

12. The system of claim 11, wherein the controller operates the system in the first mode until the digested material in the digester reaches a threshold capacity, and then operates the system in the second mode to remove the digested material from the digester.

13. A dual-mode system for processing an organic material, the system comprising:
a digester that produces a digested material;
a first thermal input device operable in a first mode to heat the organic material, produce a thermally treated material, and supply the thermally treated material to the digester; and
a second thermal input device operable in a second mode to heat the digested material from the digester and produce a useful product.

14. The system of claim 13, wherein the first and second thermal input devices operate simultaneously in the first and second modes, respectively.

15. The system of claim 13, further comprising a dewatering device downstream of the second thermal input device.

16. The system of claim 13, further comprising a third thermal input device that is selectively operable in the first mode or the second mode.

17. The system of claim 13, further comprising a second digester downstream of the digester of claim 13 and upstream of the second thermal input device, wherein the digester of claim 13 receives the thermally treated material from the first thermal input device while the second digester simultaneously directs the digested material to the second thermal input device.

18. The system of claim 13, wherein the useful product comprises at least one of a fertilizer and a solid fuel.

19. A method for processing an organic material, the method comprising the steps of:
pre-treating the organic material by heating and pressurizing the organic material in a system, the pre-treating step generating a pre-treated material;
digesting the pre-treated material to produce methane, the digesting step generating a digested material; and
post-treating the digested material by heating and pressurizing the digested material in the system to produce a useful product.

20. The method of claim 19, wherein the system operates in a first mode during the pre-treating step and in a second mode during the post-treating step.

21. The method of claim 19, wherein the pre-treating and post-treating steps occur simultaneously.

22. The method of claim 19, wherein the temperature and pressure of the post-treating step exceeds the temperature and pressure of the pre-treating step.
